(19) United States
(12) Patent Application Publication
(10) Pub. No.: US 2024/0180527 A1
(43) Pub. Date: Jun. 6, 2024

(54) ULTRASOUND DIAGNOSTIC APPARATUS AND IMAGE DATA PROCESSING METHOD

(71) Applicant: FUJIFILM Healthcare Corporation, Kashiwa-shi (JP)

(72) Inventor: Fumie Matsuda, Chiba (JP)

(21) Appl. No.: 18/518,876

(22) Filed: Nov. 24, 2023

(30) Foreign Application Priority Data
Dec. 5, 2022 (JP) 2022-194362

(51) Int. Cl. A61B 8/08; A61B 8/00

(52) U.S. Cl. CPC A61B 8/5207; A61B 8/463; A61B 8/469

(57) ABSTRACT

An image data group used in image comparison can be early acquired in an ultrasound examination. An acquisition section starts background acquisition processing of acquiring an image data group obtained in a past examination of a subject from a server after a start of an ultrasound examination and before a start of image comparison. The display processing section juxtaposes and displays a current image as a real-time ultrasound image and a past image (reference image) based on image data selected from within the image data group. In the background acquisition processing, acquisition of still image data is prioritized over acquisition of moving image data and volume data.

### CROSS-REFERENCE TO RELATED APPLICATIONS

The present application claims priority from Japanese Patent Application No. 2022-194362 filed on Dec. 5, 2022, the content of which is hereby incorporated by reference into this application.

### BACKGROUND OF THE INVENTION

### 1. Field of the Invention

The present disclosure relates to an ultrasound diagnostic apparatus and an image data processing method, and particularly, to acquisition and processing of image data.

### 2. Description of the Related Art

An ultrasound diagnostic apparatus is used in an ultrasound examination. The ultrasound diagnostic apparatus is an apparatus that forms an ultrasound image by transmitting an ultrasound wave to an inside of a body of a subject and receiving a reflected wave from the inside of the body of the subject. The ultrasound diagnostic apparatus is connected to a server via a network as necessary.

The server stores image data generated by the ultrasound diagnostic apparatus and image data generated by another medical apparatus (another examination apparatus). Examples of the other medical apparatus include a CT apparatus used in a computed tomography (CT) examination, an MRI apparatus used in a magnetic resonance imaging (MR) examination, and the like. Hereinafter, an image generated by the MRI apparatus will be referred to as an MR image.

Examples of the image data generated by the examination apparatus include still image data, moving image data, and volume data. The moving image data is composed of a plurality of pieces of frame data arranged temporally. The volume data is composed of a plurality of pieces of frame data (a plurality of pieces of slice data) arranged spatially.

In general, the ultrasound diagnostic apparatus has a function (image comparison function) of performing display processing for image comparison. By executing the image comparison function, a current image as a real-time ultrasound image and a past image (reference image) which is an image corresponding to the current image and has been acquired in the past are displayed side by side. The past image is, for example, an ultrasound image, a CT image, or an MR image. Usually, the ultrasound examination is composed of a plurality of processes. These processes include one or a plurality of processes in which the image comparison is performed.

Conventionally, one or a plurality of past examinations of the subject are specified at a point in time when an instruction to start the image comparison is issued or at a point in time when a past image for image comparison is selected, and then an image data group obtained in the one or the plurality of past examinations is specified. In a case where the image data group is stored in the server, the image data group is transferred (downloaded) from the server to the ultrasound diagnostic apparatus. In a case where the image data group is composed of a large number of pieces of image data or in a case where the moving image data or the volume data is included in the image data group, it takes a considerable amount of time to complete the acquisition of the image data group. A thumbnail image group for image selection is generated based on the image data group. In a case where the acquisition of the image data group is delayed, the display of the thumbnail image group may be delayed.

WO2018/047910A discloses a system composed of an ultrasound diagnostic apparatus and a workstation. A current image and a past image are displayed on the workstation. WO2018/047910A does not describe the transfer of the image data group from the workstation to the ultrasound diagnostic apparatus, and in particular, does not describe the transfer of the image data group before the start of the image comparison.

JP2008-183245A and JP2008-188163A disclose an ultrasound diagnostic apparatus having an image comparison function. JP2008-183245A and JP2008-188163A do not describe the earlier transfer of the image data group before the start of the image comparison.

### SUMMARY OF THE INVENTION

In a case where the acquisition of the image data group from the server is started at the start or after the start of the image comparison, a timing of completing the acquisition of the image data group is delayed. This causes a problem where the display of the thumbnail image group based on the image data group is delayed, and consecutively, the display of the past image is delayed. Such delays lead to a reduction in the efficiency of the ultrasound examination.

An object of the present disclosure is to accelerate a timing of completing acquisition of an image data group used in image comparison. Alternatively, an object of the present invention is to ensure that a part or all of a thumbnail image group is promptly displayed after the start of the image comparison.

According to an aspect of the present disclosure, there is provided an ultrasound diagnostic apparatus comprising: an acquisition section configured to acquire an image data group obtained in a past examination for a subject from a server in a current ultrasound examination for the subject; and a display processing section configured to, in a case where specific image data is selected from within the image data group in the current ultrasound examination, execute display processing of displaying a current image, which is a real-time ultrasound image, and a past image generated based on the specific image data, in which the acquisition section is configured to start background acquisition processing of acquiring the image data group from the server based on subject information specified at a start of the current ultrasound examination, prior to a start of the display processing.

According to another aspect of the present disclosure, there is provided an image data processing method comprising: a step of acquiring an image data group obtained in a past examination for a subject from a server in a current ultrasound examination for the subject; and a step of executing, in a case where specific image data is selected from within the image data group in the current ultrasound examination, display processing of displaying a current image, which is a real-time ultrasound image, and a past image generated based on the specific image data, in which background acquisition processing of acquiring the image data group from the server based on subject information specified at a start of the current ultrasound examination is started prior to a start of the display processing.

According to the aspects of the present disclosure, the completion of the acquisition of the image data group used in the image comparison is accelerated. Alternatively, according to the aspects of the present disclosure, a part or all of the thumbnail image group can be promptly displayed after the start of the image comparison.

### BRIEF DESCRIPTION OF THE DRAWINGS

FIG. 1 is a block diagram showing an examination system according to an embodiment.

FIG. 2 is a diagram showing an image for setting an extraction condition.

FIG. 3 is a diagram showing an initial screen.

FIG. 4 is a diagram showing selection of a past examination.

FIG. 5 is a diagram showing a thumbnail image column.

FIG. 6 is a diagram showing an execution state of image comparison.

FIG. 7 is a diagram showing a first example of a thumbnail image displayed during download.

FIG. 8 is a diagram showing a second example of the thumbnail image displayed during download.

FIG. 9 is a diagram showing a modification example of the thumbnail image displayed during download.

FIG. 10 is a timing chart showing an operation example of an ultrasound diagnostic apparatus.

FIG. 11 is a flowchart showing the operation example of the ultrasound diagnostic apparatus.

FIG. 12 is a diagram showing an example of a first management table (examination management table).

FIG. 13 is a diagram showing an example of a second management table (process management table).

FIG. 14 is a diagram showing an example of a third management table (image management table).

### DESCRIPTION OF THE PREFERRED EMBODIMENTS

Hereinafter, an embodiment will be described with reference to the drawings.

### (1) Outline of Embodiment

An ultrasound diagnostic apparatus according to the embodiment includes an acquisition section and a display processing section. The acquisition section acquires an image data group obtained in a past examination for a subject from a server in a current ultrasound examination for the subject. The display processing section executes, in a case where specific image data is selected from within the image data group in the current ultrasound examination, display processing of displaying a current image, which is a real-time ultrasound image, and a past image generated based on the specific image data. The acquisition section starts background acquisition processing of acquiring the image data group from the server based on subject information specified at a start of the current ultrasound examination, prior to a start of the display processing.

With the above configuration, the background acquisition processing is started prior to the start of the display processing for image comparison, so that the completion of the acquisition of the image data group can be accelerated. Therefore, the start of the display of the thumbnail image group generated based on the image data group can be accelerated. In a case where the acquisition of the image data group is completed before the start of the display processing, the delay in the display of the thumbnail image group and the delay in the display of the past image do not occur.

In the embodiment, the image data group is composed of at least one image data. The past examination includes an ultrasound examination, a CT examination, an MRI examination, and the like. In the embodiment, in a case where the image data group includes first image data having a small amount of data and second image data having a large amount of data, the acquisition of the first image data is prioritized over the acquisition of the second image data. As a result, a timing of displaying a part of the thumbnail image group can be accelerated even in a case where the background acquisition processing is not completed at the start of the image comparison.

In the embodiment, the subject information is information input to an initial image which is displayed at the start of the current ultrasound examination. With this configuration, the background acquisition processing can be automatically executed using the information input at the start of the ultrasound examination. There is no burden occurring on a user to execute the background acquisition processing. The information input to the initial image is usually performed manually, but it may be performed automatically.

In the embodiment, an extraction condition is designated before the start or at the start of the current ultrasound examination. The acquisition section acquires an image data group generated in the past examination for the subject and satisfying the extraction condition from the server as the above image data group. With this configuration, the image data group to be acquired can be narrowed down. As a result, acquisition of unnecessary image data can be avoided. In the embodiment, narrowing down the image data group is performed by the ultrasound diagnostic apparatus. For example, the image data group that satisfies the extraction condition is narrowed down through a step-by-step inquiry to the server. Narrowing down the image data group may be performed on the server.

In the embodiment, the extraction condition includes at least one of a condition for limiting a timing of the past examination, a condition for limiting a medical apparatus used to perform the past examination, or a condition for limiting a tissue examined in the past examination. The extraction condition may include other conditions.

In the embodiment, the acquisition section starts, in a case where the image data group to be acquired from the server includes still image data and moving image data, acquisition of the still image data in the background acquisition processing prior to a start of acquisition of the moving image data. With this configuration, the timing of displaying a thumbnail image corresponding to the still image data can be accelerated. In other words, the acquisition of the moving image data consisting of a large number of pieces of frame data can be postponed.

In the embodiment, the acquisition section starts, in a case where the image data group to be acquired from the server includes still image data and volume data, acquisition of the still image data in the background acquisition processing prior to a start of acquisition of the volume data. With this configuration, the timing of displaying the thumbnail image corresponding to the still image data can be accelerated. In other words, the acquisition of the volume data consisting of a large number of pieces of frame data (a large number of pieces of slice data) can be postponed.

In the embodiment, the acquisition section starts, in a case where the image data group to be acquired from the server includes the volume data, acquisition of representative frame data within the volume data in the background acquisition processing prior to the start of the acquisition of the volume data. The display processing section generates a thumbnail image representing the volume data based on the representative frame data. With this configuration, for the volume data, the timing of displaying the thumbnail image representing the volume data can be accelerated.

In the embodiment, the image data group includes moving image data. The display processing section displays information indicating that data is being acquired in a thumbnail image display region corresponding to the moving image data during acquisition of the moving image data. With this configuration, the user can be notified that the acquisition of the moving image data has not been completed yet.

In the embodiment, the information indicating that data is being acquired includes information indicating the number of pieces of frame data constituting the moving image data. With this configuration, it is possible to provide a user (a doctor, an examination technician, or the like) with an estimate of the time required for downloading moving image data. The information indicating that data is being acquired may be displayed during the download of the still image data.

In the embodiment, the display processing section displays information indicating that data is being acquired on or near a thumbnail image based on the representative frame data during acquisition of the volume data. With this configuration, the user can be notified that the acquisition of the volume data has not been completed yet.

In the embodiment, the information indicating that data is being acquired includes information indicating the number of pieces of acquired frame data. With this configuration, the user can be notified of the download progress of the volume data.

A data processing method according to the embodiment includes an acquisition step and a display processing step. In the acquisition step, an image data group obtained in a past examination for a subject is acquired from a server in a current ultrasound examination for the subject. In the display processing step, in a case where specific image data is selected from within the image data group in the current ultrasound examination, display processing of displaying a current image, which is a real-time ultrasound image, and a past image generated based on the specific image data is executed. Background acquisition processing of acquiring the image data group from the server based on subject information specified at a start of the current ultrasound examination is started prior to a start of the display processing.

The above data processing method is performed in the ultrasound diagnostic apparatus. The above data processing method can be realized as a function of hardware or a function of software. In the latter case, a program for executing the above data processing method is installed on the ultrasound diagnostic apparatus via a network or a portable storage medium. The ultrasound diagnostic apparatus is an information processing apparatus and includes a storage medium for non-temporarily storing the program.

### (2) Details of Embodiment

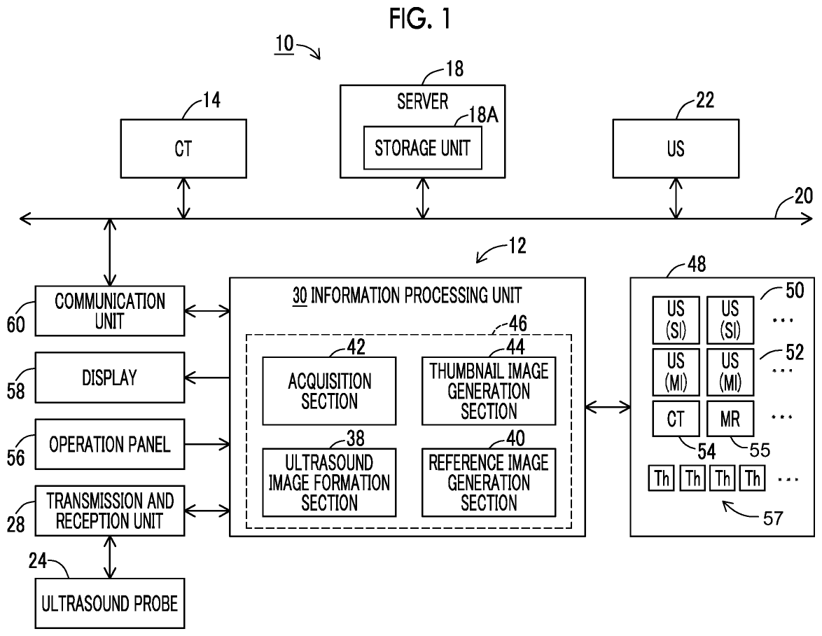

FIG. 1 shows an examination system 10 according to the embodiment. The examination system 10 is installed within, for example, a medical institution such as a hospital. In the shown example, the examination system 10 includes an ultrasound diagnostic apparatus 12, a CT apparatus 14, a server 18, and the like. The apparatuses are connected to each other via a network 20. Another ultrasound diagnostic apparatus 22 is also connected to the network 20. An MRI apparatus or the like may be connected to the network 20. In the embodiment, image data is exchanged in accordance with digital imaging and communications in medicine (DICOM), which is a standard for medical images. The server 18 is a DICOM server. Of course, the image data may be exchanged under other standards. The ultrasound diagnostic apparatus 12 has an image comparison function. The ultrasound diagnostic apparatus 12 may not have specific image data (past image data acquired from a target person of the current ultrasound examination) necessary for executing the image comparison function. In preparation for such a case, the ultrasound diagnostic apparatus 12 has a function of inquiring of the server 18 the existence or nonexistence of the specific image data and automatically acquiring the specific image data in a case where the specific image data exists on the server 18. This will be described in detail below.

The data acquired by the ultrasound diagnostic apparatuses 12 and 22 is uploaded to the server 18. Examples of the data to be uploaded include still image data and moving image data. The moving image data consists of a plurality of pieces of frame data arranged on a time axis.

CT volume data acquired by the CT apparatus 14 is uploaded to the server 18. The ultrasound volume data may be sent from the ultrasound diagnostic apparatuses 12 and 22 to the server. The volume data is three-dimensional data acquired from a three-dimensional space within an object to be examined and usually consists of a plurality of pieces of frame data (a plurality of pieces of slice data) arranged on a spatial axis. The server 18 has a storage unit 18A that stores a plurality of pieces of uploaded image data. The storage unit 18A is composed of a semiconductor memory, a hard disk, or the like.

The ultrasound diagnostic apparatus 12 includes an ultrasound probe 24. The ultrasound probe 24 includes a transducer array that transmits and receives an ultrasound wave. The transducer array is composed of a plurality of transducers arranged one-dimensionally. An ultrasound beam is formed by the transducer array, and electronic scanning is performed with the ultrasound beam. As an electronic scanning method, an electronic linear scanning method, an electronic sector scanning method, and the like are known. A two-dimensional transducer array is provided inside the ultrasound probe 24, as necessary.

A positioning system for specifying a position and an orientation of the ultrasound probe 24 may be provided. The positioning system is composed of, for example, a magnetic sensor, a magnetic field generator, and a positioning controller. In a case where the configuration is employed, the magnetic sensor is provided in the ultrasound probe 24.

A transmission and reception unit 28 is composed of a transmission unit and a reception unit. Specifically, the transmission unit is composed of an electronic circuit serving as a transmission beamformer and supplies a plurality of transmission signals in parallel to the transducer array during the transmission. As a result, a transmission beam is formed. The reception unit is composed of an electronic circuit serving as a reception beamformer and applies phase addition to a plurality of reception signals output in parallel from the transducer array during the reception. As a result, reception beam data is generated.

A beam scanning plane is formed inside the body of the subject through electronic scanning of the ultrasound beam. The beam scanning plane is a two-dimensional data acquisition region, which can be called an observation cross-section. A plurality of pieces of reception beam data arranged in an electronic scanning direction are generated during each electronic scanning of the ultrasound beam. Reception frame data is composed of the plurality of pieces of reception beam data. Each reception beam data is composed of a plurality of pieces of echo data arranged in a depth direction. By repeating the electronic scanning of the ultrasound beam, a plurality of pieces of reception frame data are sequentially generated and are sequentially sent to the information processing unit 30.

The information processing unit 30 is composed of one or a plurality of processors. For example, the information processing unit 30 is composed of a CPU that executes a program. In FIG. 1, a plurality of functions exhibited by the information processing unit 30 are represented by a plurality of blocks.

The information processing unit 30 functions as an ultrasound image formation section 38, a reference image generation section 40, an acquisition section 42, a thumbnail image generation section 44, and the like. The information processing unit 30 also functions as a display processing section 46. The display processing section 46 is a module that executes display processing for image comparison, and can also be called a display controller. In the configuration example shown in FIG. 1, the display processing section 46 includes the ultrasound image formation section 38, the reference image generation section 40, the acquisition section 42, and the thumbnail image generation section 44. The information processing unit 30 controls the operation of each component in the ultrasound diagnostic apparatus 12.

The ultrasound image formation section 38 forms a plurality of ultrasound images based on the plurality of pieces of input reception frame data. In the embodiment, a real-time tomographic image serving as a moving image is composed of the plurality of ultrasound images. Other ultrasound images may be formed. The ultrasound image formed by the ultrasound image formation section 38 is a current image.

In the embodiment, the reference image generation section 40 generates a past image as the reference image to be compared with the real-time tomographic image, that is, the current image. The past image is, for example, an ultrasound tomographic image as a still image, an ultrasound tomographic image as a moving image, a three-dimensional image based on volume data acquired by another modality (another medical apparatus), a tomographic image based on plane data extracted from the volume data, or the like.

These images are each generated based on the image data acquired in the past examination for the same subject. In the image comparison, the current image obtained in the current ultrasound examination for a specific subject and the past image obtained in the past examination for the specific subject are simultaneously displayed. The past examination includes an ultrasound examination, a CT examination, an MRI examination, and the like. In general, both the current image and the past image are images representing the same tissue. For example, image comparison is performed in the follow-up observation of a lesion part.

The reference image generation section 40 also has a function of generating the reference image based on the image data generated within the ultrasound diagnostic apparatus 12.

In the volume data, the position from which the above plane data is to be extracted is selected by the user or is automatically selected. A past image showing the same cross-section as the beam scanning plane may be automatically generated based on position information obtained by the positioning system.

The acquisition section 42 is a module that acquires the image data group from the server. In the embodiment, the acquisition section 42 specifies the image data group to be acquired from the server based on the subject information input at the start of the ultrasound examination. In a case where the user designates the extraction condition, the image data group that satisfies the extraction condition is specified. More specifically, the acquisition section 42 starts the background acquisition processing of the image data group after the start of the ultrasound examination and before the start of the display processing for image comparison. As a result, the timing of completing the acquisition of the entire image data group is accelerated. In addition, the burden on the user in acquiring the image data group is reduced. The background acquisition processing will be described in detail below.

The thumbnail image generation section 44 generates a thumbnail image group based on the acquired image data group. Each thumbnail image is a reduced image or a low-resolution image for selecting the past image. The thumbnail image generation section 44 also generates a thumbnail image representing the ultrasound image acquired and stored in the current ultrasound examination.

In a case where the still image data is acquired, the thumbnail image is generated based on the still image data. In a case where the moving image data is acquired, the thumbnail image is generated based on the representative frame data included in the moving image data. The representative frame data extracted from the moving image data is, for example, leading frame data. Under the DICOM standard, data is transferred in a file unit. In the embodiment, the moving image data constitutes one file.

In a case where the volume data is an acquisition target, the representative frame data (representative slice data) within the volume data is acquired early prior to the acquisition of the volume data. The representative frame data is, for example, central slice data. A thumbnail image corresponding to the volume data is generated based on the representative frame data. After the start of the acquisition of the representative frame data, the acquisition of the volume data including the representative frame data is started. Under the DICOM standard, the volume data is composed of, for example, a plurality of files having a plurality of pieces of frame data. However, the volume data may be composed of one file.

The storage unit 48 serving as a local storage unit is connected to the information processing unit 30. The storage unit 48 is composed of a semiconductor memory, a hard disk, or the like. The storage unit 48 stores a plurality of pieces of image data generated by the ultrasound diagnostic apparatus 12 and also stores a plurality of pieces of image data acquired from the server 18. In FIG. 1, the image data group acquired from the server 18 is shown in the storage unit 48. The image data group is composed of a plurality of pieces of ultrasound still image data 50, a plurality of pieces of ultrasound moving image data 52, CT volume data 54, MR volume data 55, and the like.

The storage unit 48 also stores a plurality of pieces of thumbnail image data 57 generated by the thumbnail image generation section 44. The storage unit 48 further stores a plurality of management tables, which will be described below.

An operation panel 56, a display 58, a communication unit 60, and the like are connected to the information processing unit 30. The operation panel 56 is an input device that includes a plurality of switches, a trackball, a keyboard, and the like. The display 58 is composed of a liquid crystal display, an organic EL display, or the like.

The display 58 juxtaposes and displays the current image and the past image during the execution of the image comparison. The current image is, for example, a tomographic image as a real-time ultrasound image. The current image is basically a moving image, but becomes a still image at the time of freeze (when transmission/reception is stopped). The past image is, for example, a tomographic image as an ultrasound image obtained in the past examination. The past image is a still image or a moving image. As the reference image, a tomographic image based on the plane data extracted from the volume data obtained in the past examination may be displayed, or a three-dimensional image based on the volume data obtained in the past examination may be displayed. Two or more past images may be simultaneously displayed.

The information processing unit 30 is connected to the network 20 via the communication unit 60. The network 20 is, for example, a LAN. The network 20 may include the Internet.

Hereinafter, the acquisition of the image data group and image processing for image comparison will be described in detail.

In the embodiment, a subject ID of the subject to which the current ultrasound examination is applied is specified at the start of the current ultrasound examination. Then, the image data group is searched using the subject ID. The image data group specified as a result of the search is composed of a plurality of pieces of image data obtained in one or a plurality of past examinations applied to the same subject.

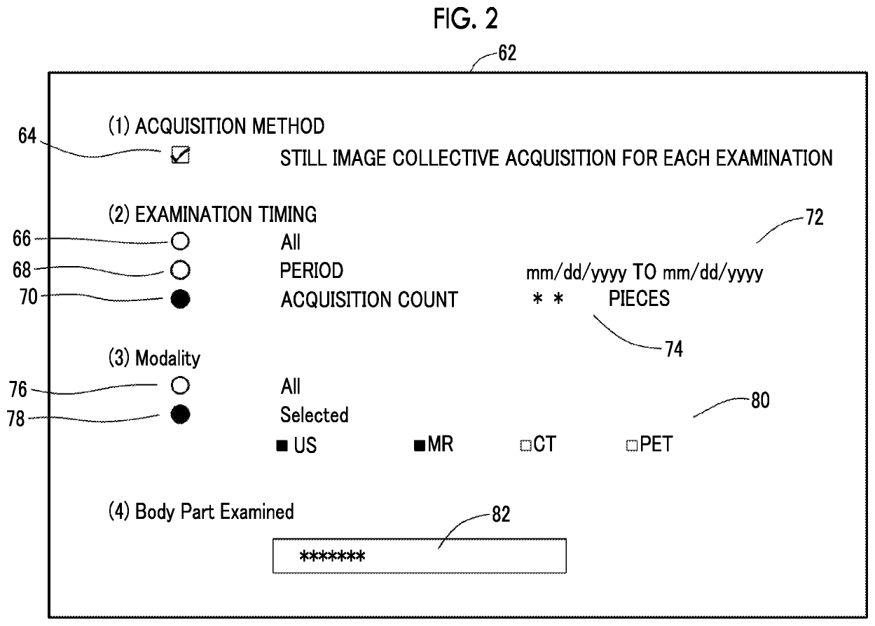

FIG. 2 shows an image 62 for designating the extraction condition. In a case where it is desired to further narrow down the image data group, the user designates the extraction condition for the narrowing down through the image 62.

In the background acquisition processing, the acquisition of the still image data is prioritized over the acquisition of the moving image data and the volume data. Under that premise, in a case where it is desired to further accelerate the acquisition completion of a set of still image data for each past examination, an input is made to a checkbox 64. That is, the presence or absence of the input to the checkbox 64 allows for a switch in the acquisition method of the image data group. Specifically, in a case of selecting still image collective acquisition for each examination, an input is made to the checkbox 64. In a case of selecting the still image collective acquisition for each of the processes constituting each examination, the input to the checkbox 64 is omitted.

Buttons 66 and 68 are operated in a case of designating a condition related to an examination timing. For example, in a case where the button 66 is turned ON, a plurality of pieces of image data acquired in all the past examinations for the subject to which the current ultrasound examination is applied are acquired. In a case where the button 68 is turned ON, a plurality of pieces of image data acquired in the past examinations within the designated period for the subject to which the current ultrasound examination is applied are acquired. Reference numeral 72 indicates a designated period. The start and end dates of the designated period are designated by the user. A button 70 is operated in a case where it is desired to designate a maximum value for the number of pieces of image data to be retroactively acquired, instead of defining the designated period. Reference numeral 74 indicates the maximum value designated by the user. By operating the button 70, a designated number of pieces of image data which are retroactively acquired with the maximum value, including image data acquired in the most recent past examination, are acquired.

Buttons 76 and 78 are operated in a case of designating a condition related to the modality (medical apparatus) used in the past examination. In a case where the button 76 is operated, image data generated in all the modalities is a search target. In a case where the button 78 is operated, image data generated with the modality designated by the user is a search target. A plurality of buttons 80 are for designating modalities.

An input field 82 is used in a case of designating a condition related to a tissue (examination part). A tissue is designated using the input field 82. The examination part may be automatically specified based on a parameter set selected during the ultrasound examination or a parameter set selected in advance.

In the background acquisition processing, the above acquisition section first searches for an image data group that satisfies both a subject condition and the extraction condition and then actually acquires the image data group specified by the search from the server.

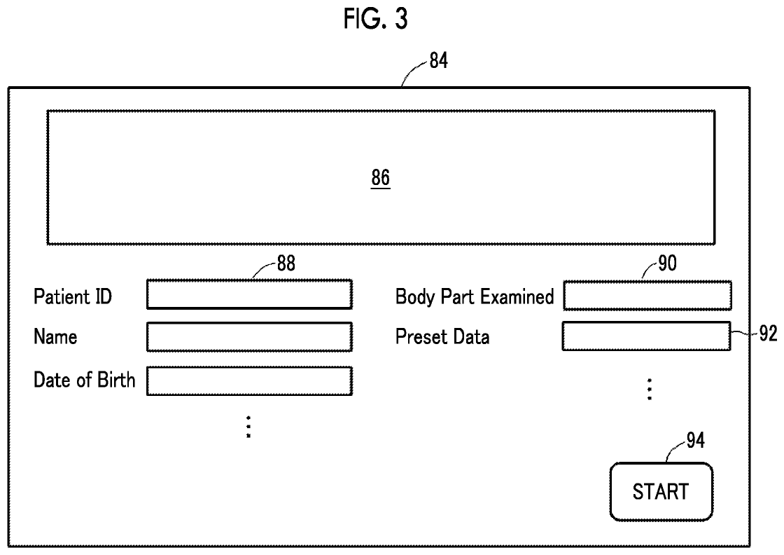

FIG. 3 shows an initial screen 84 displayed at the start of the ultrasound examination. Various types of information regarding the subject are input (registered) through the initial screen 84. An input field 88 is a field for inputting the subject ID. An input field 90 is a field for inputting the examination part. An input field 92 is a field for designating a preset as the parameter set. The parameter set is composed of a large number of parameters that define operation conditions of the ultrasound diagnostic apparatus.

The input subject ID is used in the search of the image data group. All or a part of the extraction conditions are designated based on the input contents of the input field 90 and the input field 92. An image search is performed as necessary. In that case, the search result is displayed in a search field 86. For example, a past examination list or a subject list is displayed in the search field 86. By selecting a specific item within the past examination list or selecting a specific item within the subject list, the subject ID is automatically specified, and the subject ID is automatically input to the input field 88.

The initial screen 84 includes a start button 94. In a case where the necessary input is completed and then the start button 94 is operated, transmission and reception of the ultrasound waves are started, and the background acquisition processing is started. In the background acquisition processing, prior to the execution start of the image comparison, an image data group that may be displayed during the execution of the image comparison, specifically, an image data group that satisfies the subject condition and the extraction condition, is acquired. At a point in time of the start of the image comparison, a case where the entire image data group has been acquired (a case of completed acquisition) and a case where only a part of the image data group has been acquired (a case of acquisition in progress) may occur. In any case, with the background acquisition processing, the timing of completing the acquisition of the image data group can be accelerated.

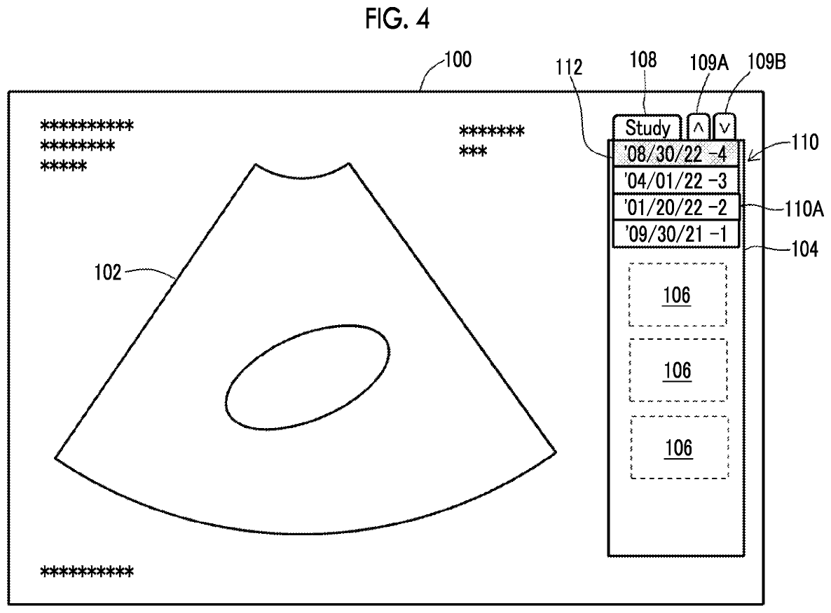

FIG. 4 shows a screen 100 of the display during the ultrasound examination. An ultrasound image 102 is displayed on the screen 100. The ultrasound image 102 is, for example, a tomographic image as a moving image. The ultrasound image 102 is a real-time image showing the inside of the body of the specific subject to which the current ultrasound examination is applied, that is, the current image.

The screen 100 includes an area 104 for displaying a thumbnail image list. The thumbnail image list is composed of, for example, a plurality of thumbnail images 106 representing a plurality of images acquired and stored in the current ultrasound examination. Buttons 109A and 109B are buttons for switching pages.

In a case where a button 108 is operated prior to the image comparison, an examination list 110 is displayed. The examination list 110 consists of a plurality of items 110A indicating a plurality of examinations performed in the past. Here, a specific item 112, that is, a specific past examination, is selected.

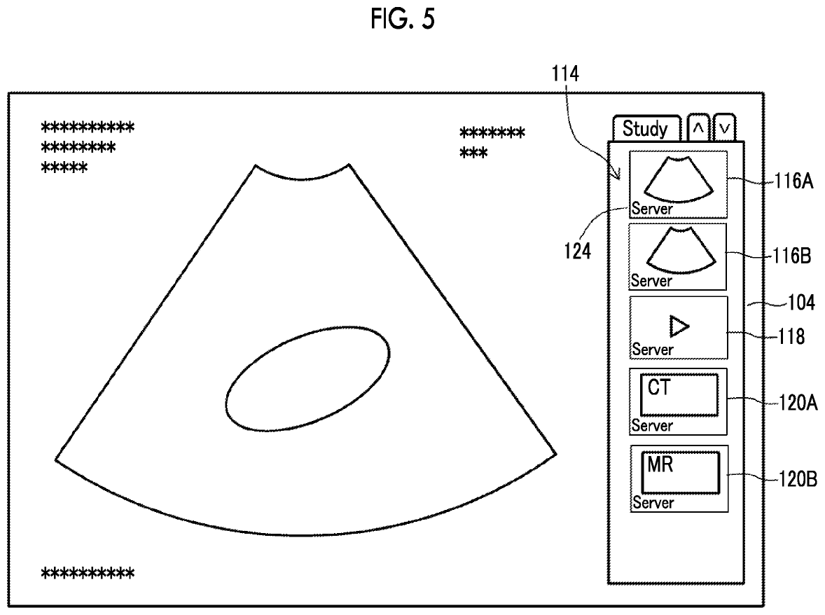

With the above selection, as shown in FIG. 5, a thumbnail image list 114 is displayed within the area 104. In a case where the acquisition of the entire image data group has been completed at a point in time of the display start of the thumbnail image list 114, the thumbnail image list 114 without missing is displayed. On the other hand, in a case where only a part of the image data group has been acquired and a remaining part has not been acquired at the point in time of the display start of the thumbnail image list 114, one or a plurality of thumbnail images corresponding to the remaining part are not displayed or one or a plurality of images instead of the one or the plurality of thumbnail images corresponding to the remaining part are displayed. This will be described below.

The thumbnail image list 114 shown in FIG. 5 is composed of a plurality of thumbnail images 116A, 116B, 118, 120A, and 120B representing a plurality of images acquired in the selected specific past examination.

Specifically, the thumbnail images 116A and 116B each represent an ultrasound still image. The thumbnail image 118 represents an ultrasound moving image. In reality, the thumbnail image 118 represents leading frame data of the ultrasound moving image. Within the thumbnail image 118, a symbol indicating that the image is a moving image is shown. The thumbnail images 120A and 120B each represent volume data. Specifically, the thumbnail image 120A represents CT volume data. The thumbnail image 120B represents MR volume data. Each thumbnail image includes a label indicating an acquisition source of the image data. For example, a label 124 indicates that the acquisition source is Server.

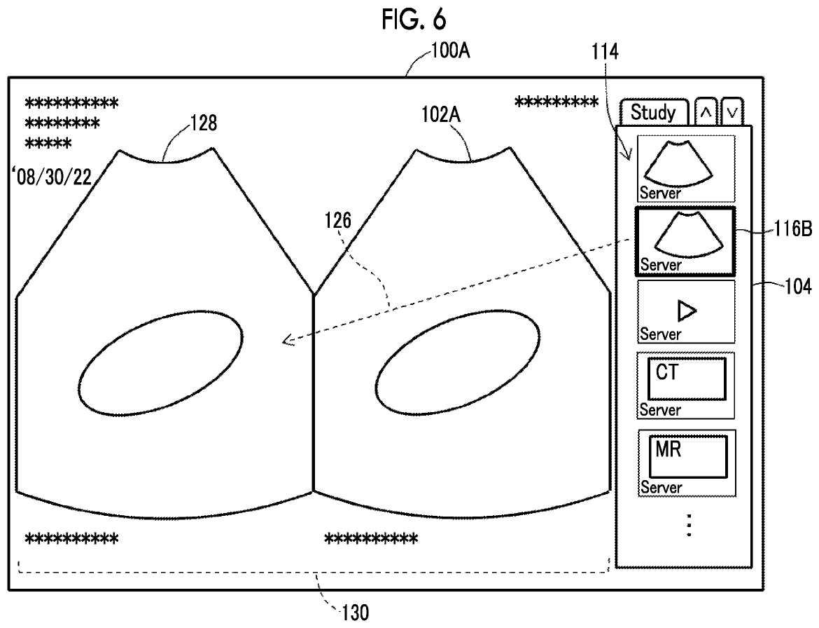

As shown in FIG. 6, in the thumbnail image list 114, in a case where the user selects the specific thumbnail image 116B, a display format is switched from a single image display format to a multi-image display format. That is, the execution of the image processing for image comparison is started. A current image 102A as an ultrasound image (moving image) and a selected past image 128 are displayed on a screen 100A. Reference numeral 126 indicates a relationship between the thumbnail image 116B and the past image 128.

The user evaluates and diagnoses the current image 102A while referring to the past image 128. The screen 100A includes an area 130 for displaying a plurality of images and the area 104 for displaying the thumbnail image list.

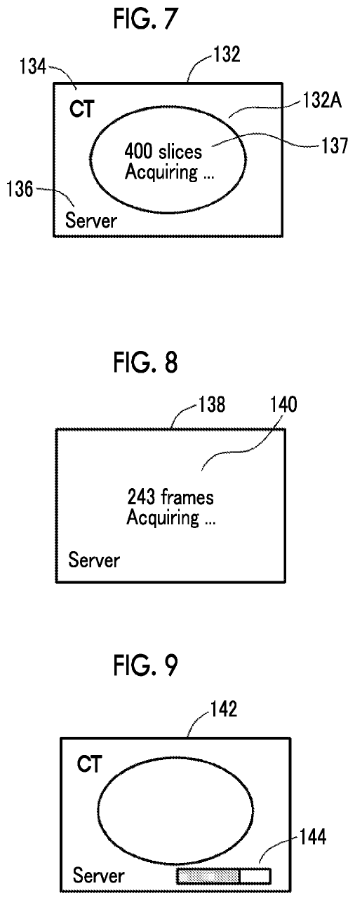

FIG. 7 shows a first example of the thumbnail image displayed during download. A thumbnail image 132 represents CT volume data and specifically, is displayed in a case where acquisition of the CT volume data has not been completed.

The thumbnail image 132 is generated based on the representative frame data in the CT volume data and includes a tissue image 132A. A label 134 indicates that the image is a CT image. A label 136 indicates the acquisition source of the image data. Information 137 displayed in the central portion of the thumbnail image 132 is a description indicating that the CT image data (volume data) is being downloaded. The information 137 includes information indicating the number of pieces of frame data constituting the volume data.

FIG. 8 shows a second example of the thumbnail image displayed during download. A thumbnail image 138 corresponds to the ultrasound moving image and the download of the moving image data has not been completed. The thumbnail image 138 is a low-resolution image having no tissue image and is a pseudo thumbnail image or an alternative thumbnail image. Information 140 indicating that the image data is being downloaded is displayed in the central portion of the thumbnail image 138. The information 140 includes information indicating the number of pieces of frame data constituting the moving image. The thumbnail image 138 is displayed in an area where a regular thumbnail image displayed after the completion of the download of the moving image data is displayed.

FIG. 9 shows a modification example of the thumbnail image displayed during download. A thumbnail image 142 includes a graph showing the download progress. The download progress may be represented by other display forms. In the embodiment, in addition to the information indicating that the volume data is being downloaded and the information indicating that the moving image data is being downloaded, the information indicating that the still image data is being downloaded is also displayed.

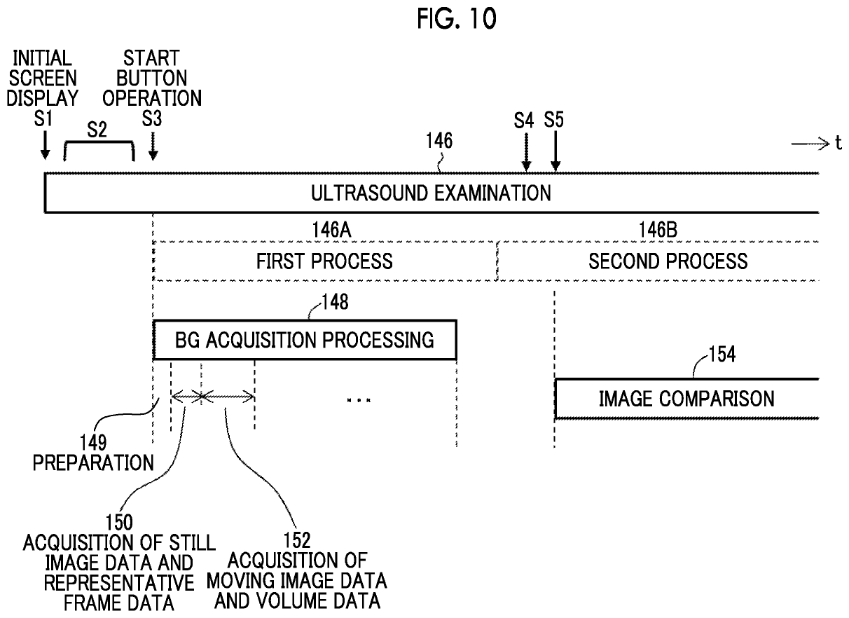

FIG. 10 shows an operation example of the ultrasound diagnostic apparatus as a timing chart. The horizontal axis is the time axis. Reference numeral 146 indicates an ultrasound examination. An initial screen is displayed in S1, and information such as a subject ID is input through the initial screen in S2. In S3, the start button is operated. As a result, transmission and reception are started, and background acquisition processing 148 is started. The ultrasound examination 146 consists of a plurality of processes that are continuous in chronological order. In FIG. 10, a first process 146A and a second process 146B are shown. The first process 146A is, for example, a process of examining a first tissue, and the second process 146B is a process of examining a second tissue.

In the background acquisition processing 148, first, in a preparation period 149, information for specifying one or a plurality of past examinations for the subject is acquired from the server. An image data group that satisfies the extraction condition is specified based on the information.

In a case where the still image collective acquisition for each examination is designated as the acquisition method, for example, past examinations are retroactively and sequentially selected, and first processing 150 and second processing 152 are performed for each selected examination. In the first processing, one or a plurality of pieces of single frame data are acquired. Each single frame data is still image data or representative frame data. In the second processing, one or a plurality of pieces of multi-frame data are acquired. Each multi-frame data is moving image data or volume data.

Meanwhile, in a case where the still image collective acquisition for each process is designated as the acquisition method, processes constituting each past examination are sequentially selected, and the first processing 150 and the second processing 152 are performed for each selected process.

In S4, the past examination list is displayed, and a specific examination is selected from within the past examination list. As a result, a thumbnail image list indicating a plurality of images acquired in the specific examination is displayed. In S5, a specific thumbnail image is selected from within the thumbnail image list. That means the selection of the past image. As a result, image comparison 154 is executed. That is, the current image and the past image are juxtaposed and displayed.

In the example shown in FIG. 10, the acquisition of the image data group is completed before the execution start of the image comparison 154, that is, the background acquisition processing is completed. In that case, the thumbnail image list is displayed promptly after the selection of the past examination. Therefore, the delay in the display of the past image does not occur. Since the background acquisition processing is started immediately after the start button operation (S3) in the embodiment even in a case where the acquisition of the entire image data group has not been completed at the point in time of the start of the image comparison, the acquisition completion timing of the image data group can be significantly accelerated as compared with a case where the acquisition of the image data group is started after the selection of the examination (S4). In addition, since the still image data having a small amount of data is acquired early, the display start of a part of the thumbnail image list can be accelerated. As a result, the stress generated in the user can be reduced.

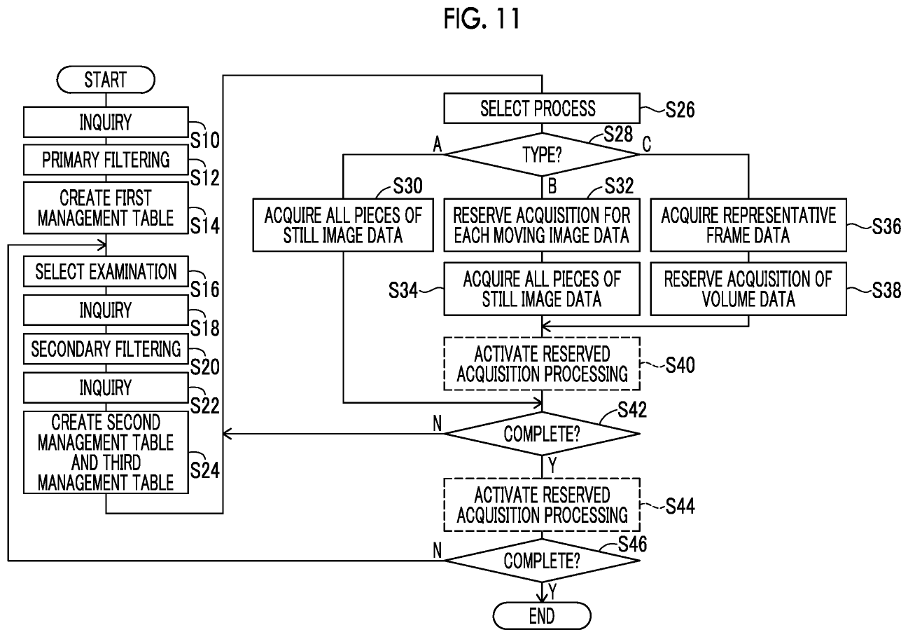

FIG. 11 shows an operation example of the ultrasound diagnostic apparatus as a flowchart. In S10, a primary inquiry from the ultrasound diagnostic apparatus to the server is performed. As a result, information for specifying one or a plurality of past examinations for the subject to which the current ultrasound examination is applied is acquired. Primary filtering is applied to the information in S12, and specifically, an examination that satisfies a timing condition is extracted in S12. In S14, a first management table is created based on the information after the primary filtering.

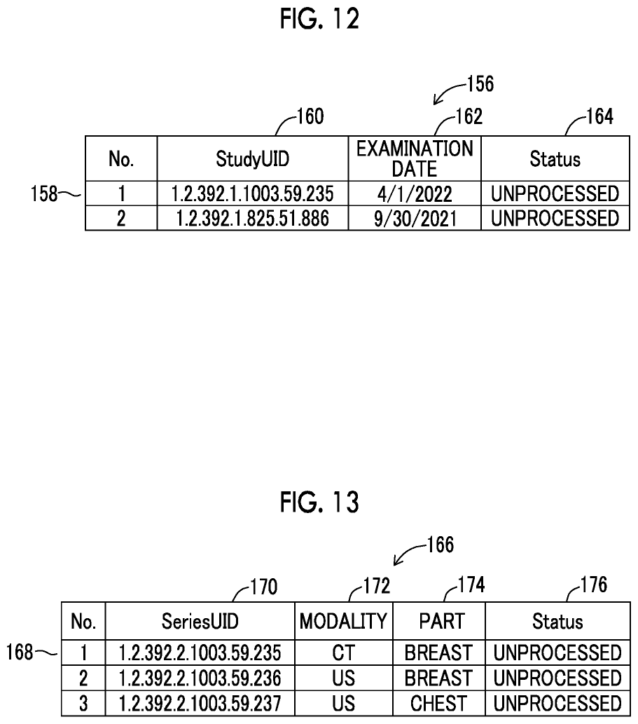

FIG. 12 shows an example of the first management table. A first management table 156 is an examination management table. The first management table 156 is composed of a plurality of records 158 corresponding to a plurality of examinations that satisfy the timing condition. Each record includes a StudyUID 160, an examination date 162, and a status 164. The StudyUID 160 is an examination identifier following DICOM. The status 164 is information indicating an acquisition status of a plurality of pieces of image data associated with the examination.

Returning to FIG. 11, in S16, one examination is selected from within the first management table. In S18, information for specifying one or a plurality of processes constituting the selected examination is acquired from the server through a secondary inquiry to the server. In S20, secondary filtering is applied to the information, and specifically, in S20, a process that satisfies the modality condition and the tissue condition is extracted. Subsequently, in S22, one or a plurality of pieces of image data associated with each process specified after the secondary filtering are specified through a tertiary inquiry to the server. In S24, a second management table and a third management table to be described below are created.

FIG. 13 shows an example of the second management table. A second management table 166 is a process management table. Here, the process is a concept corresponding to Series in DICOM and corresponds to one unit constituting the examination. The second management table 166 is composed of a plurality of records 168 corresponding to the plurality of processes. Each of the record 168 includes a SeriesUID 170, a modality 172, a part 174, and a status 176. The SeriesUID 170 is a process identifier following DICOM. The secondary filtering is executed based on the modality 172 and the part 174. The status 176 is information indicating an acquisition status of a plurality of pieces of image data associated with the process.

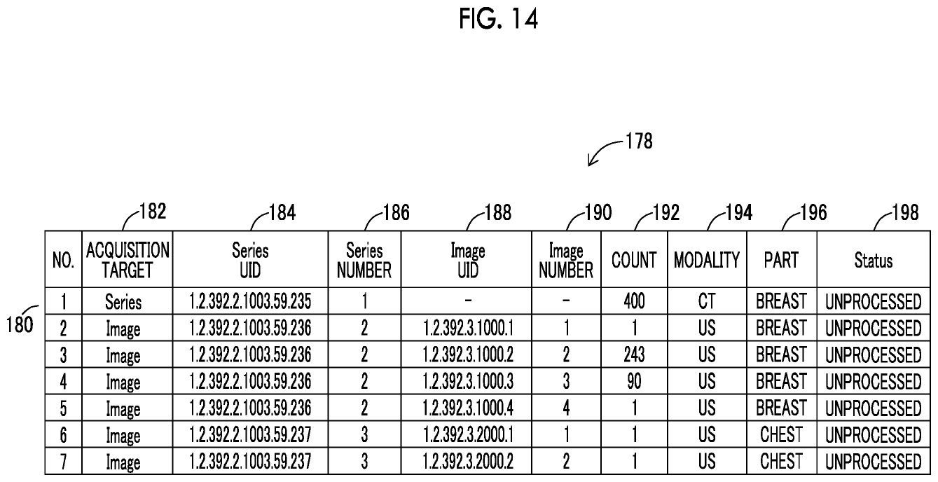

FIG. 14 shows an example of the third management table. A third management table 178 is an image management table. The third management table 178 is composed of a plurality of records 180 corresponding to a plurality of pieces of image data which are acquisition targets. Each of the record 180 includes an acquisition target 182, a SeriesUID 184, a Series number 186, an ImageUID 188, an Image number 190, a count 192, a modality 194, a part 196, and a status 198.

The acquisition target 182 is basically one image data constituting one file. It should be noted that, in the embodiment, the entire volume data is regarded as a single acquisition target. In the shown example, one volume data corresponds to one Series in accordance with DICOM. In reality, one volume data is composed of, for example, a plurality of files. The ImageUID 188 is an image identifier following DICOM. The count 192 represents the number of images constituting the acquisition target. In a case of the volume data and the moving image, the count 192 is a numerical value of 2 or more. The modality is information for specifying the medical apparatus that has generated the image data. The status 198 is information indicating an acquisition status of the image data.

Returning to FIG. 11, in S26, one process, that is, one Series, is selected from within the second management table. In S28, the type of the image data group belonging to the selected process is identified. In a case where the entire image data group is still image data (in a case of Case A), S30 is executed. In a case where the image data group is composed of both the still image data and the moving image data (in a case of Case B), S32 is executed. In a case where the image data group is composed of the volume data (in a case of Case C), S36 is executed.

In S30, the entire still image data is acquired from the server. In S32, acquisition processing is reserved for each moving image data, and in S34, the entire still image data is acquired from the server. In S36, the representative frame data within the volume data is acquired early from the server, and in S38, the acquisition processing of the volume data is reserved.

Each time the acquisition of the image data is completed, the status of the image data in the third management table is changed, and specifically, “acquired” is registered as the status. In a case where the acquisition of the representative frame data is completed, “partially acquired” is registered as the status corresponding to the representative frame data. In a case where the acquisition processing of the moving image data is reserved, “acquiring” is registered as the status corresponding to the moving image data. The same applies to a case of the volume data.

In a case where the acquisition of one or a plurality of pieces of image data belonging to the process is completed, the status of the process in the second management table is changed, and specifically, “acquired” is registered as the status. In a case where the acquisition of all the pieces of image data belonging to the examination is completed, the status of the examination is changed to “acquired”.

In a case where the still image collective acquisition for each process is designated, S40 is performed. Meanwhile, in a case where the still image collective acquisition for each examination is designated, S44 is performed. In S40, the acquisition processing is activated for each reserved acquisition processing. That is, one or a plurality of processes of sub-acquisition processing are executed in parallel to the main acquisition processing shown in FIG. 11. In S42, it is determined whether or not “acquired” has been registered for all the processes in the second management table. In a case where there is a process having an unprocessed status, the steps after S26 are executed again.

In a case where the still image collective acquisition for each examination is designated, the acquisition processing is activated for each reserved acquisition processing in S44. In a case where S44 is executed, the acquisition of all the pieces of still image data belonging to a certain examination is completed and then, the acquisition of the moving image data and the volume data belonging to the examination is started. As a result, the acquisition of the still image set is prioritized. In S46, the first management table is referred to, and it is determined whether or not “acquired” is registered for all the examinations. In a case where there is an examination having an unprocessed status, the steps after S16 are executed again.

As described above, with the ultrasound diagnostic apparatus according to the embodiment, the completion timing of acquiring the image data group used in the image comparison can be accelerated. In addition, the display start timing of the thumbnail image group after the start of the image comparison can be accelerated.

What is claimed is:

1. An ultrasound diagnostic apparatus comprising:
an acquisition section configured to acquire an image data group obtained in a past examination for a subject from a server in a current ultrasound examination for the subject; and
a display processing section configured to, in a case where specific image data is selected from within the image data group in the current ultrasound examination, execute display processing of displaying a current image, which is a real-time ultrasound image, and a past image generated based on the specific image data,
wherein the acquisition section is configured to start background acquisition processing of acquiring the image data group from the server based on subject information specified at a start of the current ultrasound examination, prior to a start of the display processing.

2. The ultrasound diagnostic apparatus according to claim 1,
wherein the subject information is information input to an initial image which is displayed at the start of the current ultrasound examination.

3. The ultrasound diagnostic apparatus according to claim 1,
wherein an extraction condition is designated before the start or at the start of the current ultrasound examination, and
the acquisition section is configured to acquire an image data group generated in the past examination for the subject and satisfying the extraction condition from the server as the image data group.

4. The ultrasound diagnostic apparatus according to claim 3,
wherein the extraction condition includes at least one of a condition for limiting a timing of the past examination, a condition for limiting a medical apparatus used to perform the past examination, or a condition for limiting a tissue examined in the past examination.

5. The ultrasound diagnostic apparatus according to claim 1,
wherein the acquisition section is configured to, in a case where the image data group to be acquired from the server includes still image data and moving image data, start acquisition of the still image data in the background acquisition processing prior to a start of acquisition of the moving image data.

6. The ultrasound diagnostic apparatus according to claim 1,
wherein the acquisition section is configured to, in a case where the image data group to be acquired from the server includes still image data and volume data, start acquisition of the still image data in the background acquisition processing prior to a start of acquisition of the volume data.

7. The ultrasound diagnostic apparatus according to claim 6,
wherein the acquisition section is configured to, in a case where the image data group to be acquired from the server includes the volume data, start acquisition of representative frame data within the volume data in the background acquisition processing prior to the start of the acquisition of the volume data, and
the display processing section is configured to generate an image representing the volume data based on the representative frame data.

8. The ultrasound diagnostic apparatus according to claim 1,
wherein the image data group includes moving image data, and
the display processing section is configured to display information indicating that data is being acquired in a thumbnail image display region corresponding to the moving image data during acquisition of the moving image data.

9. The ultrasound diagnostic apparatus according to claim 8,
wherein the information indicating that data is being acquired includes information indicating the number of pieces of frame data constituting the moving image data.

10. The ultrasound diagnostic apparatus according to claim 7,
wherein the display processing section is configured to display information indicating that data is being acquired on or near a thumbnail image based on the representative frame data during the acquisition of the volume data.

11. The ultrasound diagnostic apparatus according to claim 10,
wherein the information indicating that data is being acquired includes information indicating the number of pieces of acquired frame data.

12. An image data processing method comprising:
a step of acquiring an image data group obtained in a past examination for a subject from a server in a current ultrasound examination for the subject; and
a step of executing, in a case where specific image data is selected from within the image data group in the current ultrasound examination, display processing of displaying a current image, which is a real-time ultrasound image, and a past image generated based on the specific image data,
wherein background acquisition processing of acquiring the image data group from the server based on subject information specified at a start of the current ultrasound examination is started prior to a start of the display processing.

13. A non-transitory storage medium storing a program executed in an ultrasound diagnostic apparatus, the program comprising:
a function of acquiring an image data group obtained in a past examination for a subject from a server in a current ultrasound examination for the subject; and
a function of executing, in a case where specific image data is selected from within the image data group in the current ultrasound examination, display processing of displaying a current image, which is a real-time ultrasound image, and a past image generated based on the specific image data,
wherein background acquisition processing of acquiring the image data group from the server based on subject information specified at a start of the current ultrasound examination is started prior to a start of the display processing.
